(19) United States
(12) Patent Application Publication
(10) Pub. No.: US 2026/0162791 A1
(43) Pub. Date: Jun. 11, 2026

(54) POST-ACUTE CARE RECOMMENDATION GENERATION

(71) Applicant: Post Acute Analytics, Inc., Irving, TX (US)

(72) Inventors: Fadi Almachraki, Philadelphia, PA (US); Bryan Grohman, Plano, TX (US); Cory Upton, Frisco, TX (US); Bonnie Hardik, Cottontown, TN (US); Syed Saqib Akhter, Burr Ridge, IL (US); Manideep Reddy Gangula, Hyderabad (IN)

(73) Assignee: Post Acute Analytics, Inc., Irving, TX (US)

(21) Appl. No.: 19/180,678

(22) Filed: Apr. 16, 2025

Related U.S. Application Data
(60) Provisional application No. 63/635,547, filed on Apr. 17, 2024.

(51) Int. Cl. G16H 20/00; G16H 15/00

(52) U.S. Cl. CPC G16H 20/00; G16H 15/00

(57) ABSTRACT

In certain aspects, a method for generating a post-acute care recommendation includes generating training documents. The method includes transmitting the training documents to an AI service for training to identify presence of various clinical indicators in the training documents. The method includes transmitting, to the AI service, clinical documents associated with a patient. The method includes receiving, from the AI service based on the training to identify presence of various clinical indicators in the training documents, output indicating presence of the various clinical indicators in the clinical documents. The method includes generating, based on the output indicating presence of the various clinical indicators in the clinical documents, a recommendation of whether or not the patient associated with the clinical documents should be admitted to a SNF for post-acute care.

### CROSS-REFERENCE TO RELATED APPLICATION

The present application claims the benefit of priority under 35 U.S.C. § 119 from U.S. Provisional Patent Application Ser. No. 63/635,547 entitled “Post-Acute Care Recommendation Generation,” filed on Apr. 17, 2024, the disclosure of which is hereby incorporated by reference in its entirety for all purposes.

### FIELD

This disclosure generally relates to the data analytics, and more specifically relates to generating post-acute care recommendations.

### BACKGROUND

Upon being discharged from a hospital, patients will often times require post-acute patient care, such as physical or occupational therapy, continued wound care, administration of IV antibiotics, feeding tube care, and/or the like. Post-acute patient care can take place in a number of different types of facilities or settings, including inpatient rehab facilities, skilled nursing facilities, home health settings, long-term acute care facilities, hospice, and others. A decision must be made by the hospital staff, the patient, and the patient's health insurer as to which specific care setting to transition the patient when they are discharged from the hospital. One critical decision in particular is whether to discharge the patient to a skilled nursing facility, as opposed to home discharge or discharging to another facility that would provide an appropriate alternate level of care than that provided by skilled nursing facilities. Typically, it is a manual process for health insurance providers/companies to make these decisions, whereby a hospital discharge planner working for the hospital (or similar health care provider) submits a manual authorization request to the patient's health insurance company or payor for the patient to be admitted to a skilled nursing facility. That request is reviewed by a utilization review nurse employed by the patient's health insurance company who then either approves or denies the request based on a number of different factors.

This fully manual process is suboptimal for a number of reasons. First, it is manual and time consuming. Second, it is subjective based on the various reviewers involved at different points in the process, and thus not standardized. For example, different hospital discharge planners can adopt different criteria for making the request for admission to a skilled nursing facility, and different utilization review nurses can approve or deny the requests for different clinical reasons. Accordingly, there is a need for a better method and system for efficiently making an objective recommendation or approval decision, based on standardized criteria, as to whether a patient admitted to a hospital should thereafter be discharged to a skilled nursing facility for post-acute care.

### BRIEF DESCRIPTION OF DRAWINGS

The present invention is described in detail below with reference to the attached drawing figures, wherein:

FIG. 1 illustrates an example architecture for generating a post-acute care recommendation.

FIG. 2 is a block diagram illustrating the example computing device, AI service, and electronic medical records service from the architecture of FIG. 1, according to certain aspects of the disclosure.

FIG. 3 is a flow chart showing the process for the recommendation logic used to make recommendations as to admission of a patient to a SNF for post-acute care, according to certain aspects of the disclosure.

FIG. 4 illustrates an example process for generating a post-acute care recommendation using the example computing device, AI service, and electronic medical records service of FIG. 2.

FIG. 5 illustrates another example process for generating a post-acute care recommendation using the example computing device, AI service, and electronic medical records service of FIG. 2.

FIG. 6 is a block diagram illustrating an example computer system with which the computing device and electronic medical records service can be implemented.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

While the present invention is capable of being embodied in various forms, the description below of several embodiments is made with the understanding that the present disclosure is to be considered as an exemplification of the claimed subject matter, and is not intended to limit the appended claims to the specific embodiments described herein. The headings used throughout this disclosure are provided for convenience only and are not to be construed to limit the claims in any way. The various embodiments disclosed herein may be combined with other embodiments for the creation and description of yet additional embodiments.

Throughout the specification and claims, the following terms take the meanings explicitly associated herein, unless the context clearly dictates otherwise. The phrase “in one embodiment” does not necessarily refer to the same embodiment, although it may. Furthermore, the phrase “in another embodiment” or “in an alternate embodiment” does not necessarily refer to a different embodiment, although it may. Thus, as described below, various embodiments may be readily combined without departing from the scope or spirit of the present disclosure.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The present disclosure is drawn to computer-implemented methods and analysis systems for automatically and objectively analyzing a patient's clinical documents following a stay in a hospital or other healthcare facility for acute care, and recommending whether a patient to be discharged from the hospital should thereafter be admitted to a skilled nursing facility (“SNF”) for post-acute care.

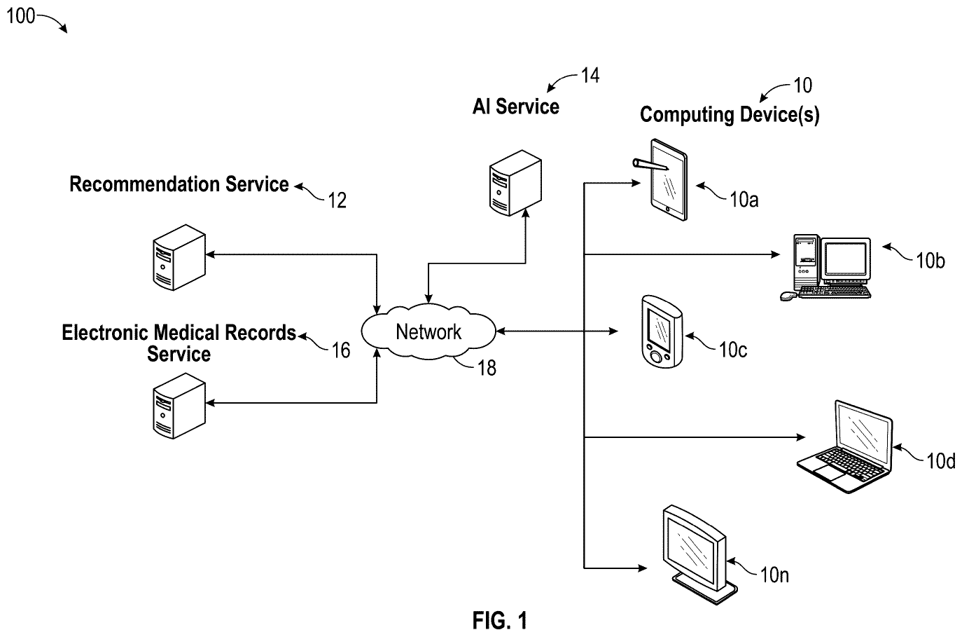

FIG. 1 illustrates an example architecture 100 for generating a post-acute care recommendation. For example, the architecture 100 includes at least one computing device 10, such as a first computing device 10a and a second computing device 10b to an nth computing device 10n, a recommendation service 12, an AI service 14, and an electronic medical records service 16 all connected over a network 18.

The recommendation service 12 can be any device having an appropriate processor, memory, and communications capability for communicating with the at least one computing device 10, such as the first computing device 10a, the AI service 14, and the electronic medical records service 16. For purposes of load balancing, the recommendation service 12 may include multiple servers.

The AI service 14 can be any device having an appropriate processor, neural network, memory, and communications capability for communicating with the recommendation service 12, the electronic medical records service 16, and, in certain aspects, the at least one computing device 10, such as the first computing device 10a.

The electronic medical records service 16 can be any device having an appropriate processor, memory, and communications capability for communicating with the recommendation service 12, the AI service, and, in certain aspects, the at least one computing device 10, such as the first computing device 10a.

The at least one computing device 10, such as the first computing device 10a, to which the recommendation service 12, the AI service 14, and the electronic medical records service 16 can communicate with over the network 18, can be, for example, a tablet computer, a mobile phone, a mobile computer, a laptop computer, a portable media player, an electronic book (eBook) reader, or any other device having appropriate processor, memory, and communications capabilities. In certain aspects, the recommendation service 12, the AI service 14, and the electronic medical records service 16 can be a cloud computing server of an infrastructure-as-a-service (IaaS) and be able to support a platform-as-a-service (PaaS) and software-as-a-service (SaaS) services.

The network 18 can include, for example, any one or more of a personal area network (PAN), a local area network (LAN), a campus area network (CAN), a metropolitan area network (MAN), a wide area network (WAN), a broadband network (BBN), the Internet, and the like. Further, the network 18 can include, but is not limited to, any one or more of the following network topologies, including a bus network, a star network, a ring network, a mesh network, a star-bus network, tree or hierarchical network, and the like.

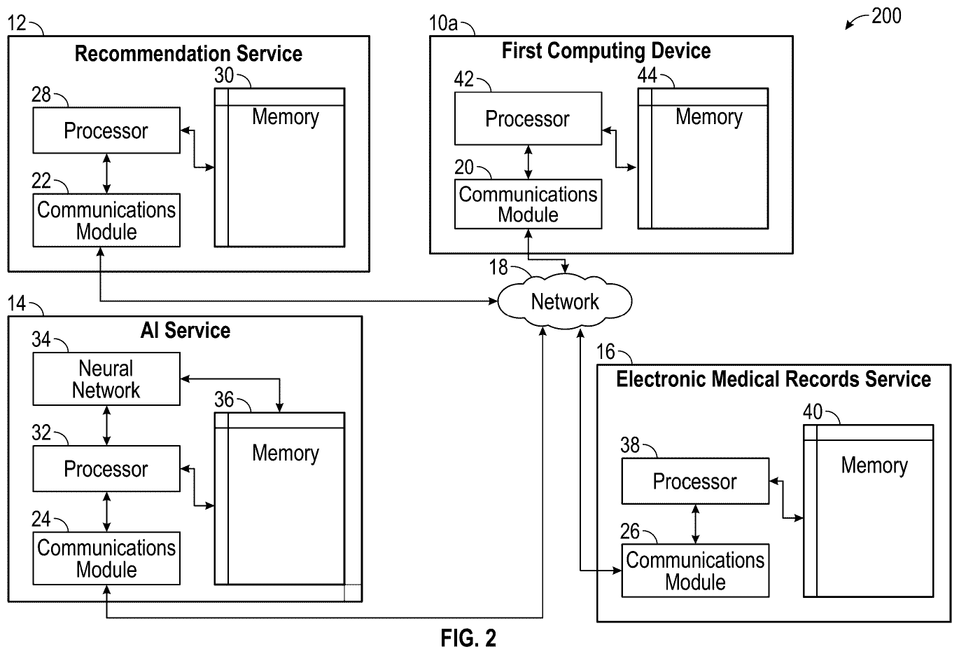

FIG. 2 is a block diagram illustrating examples of the at least one computing device 10, such as the first computing device 10a, the recommendation service 12, the AI service 14, and the electronic medical records service 16 in the architecture of FIG. 1 according to certain aspects of the disclosure. It should be understood that for purposes of explanation the first computing device 10a is described, but any number of the at least one computing device 10 could be used.

The at least one computing device 10, such as the first computing device 10a, the recommendation service 12, the AI service 14, and the electronic medical records service 16 are connected over the network 18 via respective communication modules 20, 22, 24, 26. The communication modules 20, 22, 24, 26 are configured to interface with the network 18 to send and receive information, such as data, requests, responses, and commands to other devices on the network 18. The communications modules 20, 22, 24, 26 can be, for example, modems or Ethernet cards.

The recommendation service 12 includes a processor 28, the communications module 24, and a memory 30. The processor 28 of the recommendation service 12 is configured to execute instructions, such as instructions physically coded into the processor 28, instructions received from software in the memory 30, or a combination of both. The processor 28 of the recommendation service 12 is configured to perform any of its functions described herein.

The AI service 14 includes a processor 32, the communications module 24, a neural network 34, and a memory 36. The processor 32 of the AI service 14 is configured to execute instructions, such as instructions physically coded into the processor 28, instructions received from software in the memory 36, or a combination of both. The processor 28 of the recommendation service 12 is configured to perform any of its functions described herein. The neural network 34 is configured to perform, but is not limited to, natural language processing, large language model processing, machine learning, and other appropriate processes.

The electronic medical records service 16 includes a processor 38, the communications module 26, and a memory 40. The processor 38 of the electronic medical records service 16 is configured to execute instructions, such as instructions physically coded into the processor 38, instructions received from software in the memory 40, or a combination of both. The processor 38 of the electronic medical records service 16 is configured to perform any of its functions described herein.

The at least one computing device 10, such as the first computing device 10a, includes a processor 42, the communications module 20, and a memory 44. The processor 42 of the at least one computing device 10 is configured to execute instructions, such as instructions physically coded into the processor 42, instructions received from software in the memory 44, or a combination of both. The processor 42 of the at least one computing device 10 is configured to perform any of its functions described herein.

In certain aspects, the computer-implemented method for recommending whether or not a patient should be discharged to a SNF includes, for example, the recommendation service 12 communicating with the electronic medical records service 16 associated with a hospital or acute care facility, such that the recommendation service 12 has complete data integration with patient medical records associated with the electronic medical records service 16. The method further includes developing and training the neural network 34 of the AI service 14. The neural network 34 of the AI service 14 is trained to recognize and/or document specific text, phrasing, and/or data criteria located in the clinical documents of a patient's medical records that indicates the presence or absence of various clinical indicators for that patient that either support or weigh against a recommendation of admission of that patient to a SNF. After the AI service 14 is trained, in certain aspects, the recommendation service 12 retrieves from the electronic medical records service 16 the clinical documents of a patient to be discharged from a hospital after a stay for acute care. The method continues with the recommendation service 12 transmitting to the AI service 14 for automatically searching or scanning the patient's clinical documents to identify the presence or absence of specific text, natural language phrasing, and/or data criteria contained therein, which criteria the AI service 14 has been taught is associated with or corresponds to each of a predefined set of clinical indicators, or category of clinical indicators, that either support or weigh against a recommendation of admission to a SNF. If a patient's clinical documents include any such criteria corresponding to a given clinical indicator or category thereof, then the AI service 14 applies corresponding tags or labels to the patient's clinical documents indicative that the patient's clinical documents include such clinical indicator or category of clinical indicator.

As such, the recommendation service 12 can thus scan a patient's clinical documents, receive a determination, via the AI service 14, that the presence or absence of each clinical indicator or category thereof for which the AI service 14 searched the clinical documents, and receive the results from the AI service 14 accordingly to identify each clinical indicator or category thereof that is either present in, or absent from, the documents. The documenting can be done in any number of ways, including, in one embodiment, applying tags or labels to the patient's clinical documents corresponding to each such clinical indicator found to be present in the patient's clinical documents during the patient's acute care stay in the hospital. In other aspects, the recommendation service 12 may generate a true/false table, or other data, in which is recorded each and every clinical indicator or category thereof for which the AI service 14 searched and whether such indicator or category was found in the clinical documents of the patient's medical records.

The presence or absence of each such clinical indicator in the patient's medical records are factors that either support or weigh against a recommendation that the patient be admitted to a SNF for post-acute care upon discharge from the hospital or other healthcare facility. The recommendation service 12 next executes a recommendation logic program that outputs a final recommendation to the patient's insurer, payor, or other such interested party as to whether or not the patient should be admitted to SNF for post-acute care. The outputted recommendation is made by, for example, a recommendation logic program of the recommendation service 12 based on the presence or absence of one or more of the clinical indicators in the patient's clinical documents. With this methodology, which is data driven and based on the information contained in a patient's clinical records, the recommendation service 12 is able to use standardized criteria for the entire patient population to quickly, efficiently, and objectively output a recommendation to the patient's insurer, payor, or other party as to whether or not the patient should be admitted to a SNF for post-acute care.

### Building/Training the AI Model

To build or train the AI service 14, a set of training documents are transmitted by the recommendation service 12 as input to the AI service 14 so that the AI service 14 can learn both the criteria it will be searching for within a patient's clinical documents, and the significance or meaning of each such criteria. In this regard, in one embodiment, the AI service 14 may be a natural language processing model. In certain aspects, a large number of clinical documents from patient records of prior acute care patient stays are first annotated by the recommendation service 12, and then subsequently loaded or transmitted to the learning processor (e.g, the neural network 34) of the AI service 14 for use as the training documents. In certain aspects, the learning processor of the AI service 14 will then scan the annotated text of the training documents to learn to identify, from the annotations, the specific text, phrases, and/or data that corresponds to the presence of various clinical indicators in the training documents. The presence or absence of these various clinical indicators are the factors that will be used by the recommendation logic of the recommendation service 12 to make a recommendation as to admission of a patient to a SNF for post-acute care.

The clinical documents from patient medical records that are to be used as inputs for training the AI model can include, for example, one or more of physician/clinician notes, nursing notes, therapy notes, lab results, and other appropriate inputs. In certain aspects, for example, about 400 clinical documents from prior acute patient stays in hospitals are used to train the AI service 14. In other aspects, there may be 50, 100, 150, 200, 250, 300, 450, 500, or any other appropriate number, clinical documents used as input to train the AI service 14. The more documents and data that can be used to train the AI service 14, the better the AI service 14 will be at recognizing the language, text, phrasing, and/or data present in a patient's clinical documents that correspond to a given clinical indicator and for allowing the recommendation service 12 to provide a standardized and objectively appropriate recommendation regarding admission of a patient to a SNF for post-acute care.

For the AI service 14 to be able to learn what it is searching for, in indicating the presence of any given clinical indicator in a patient's medical records, the clinical documents retrieved by the recommendation service 12 and used to train the AI service 14 are first annotated by the recommendation service 12 to identify text therein that corresponds to each specific clinical indicator that either supports or weighs against admission of a patient to a SNF for post-acute care. In certain aspects, the annotation can be performed, via the recommendation service 12, on the at least one computing device 10 by clinical experts, and utilizes appropriate technology, such as annotation software, for example, that will permit the clinical experts to review, on the at least one computing device 10 via the recommendation service 12, each such training document and digitally/electronically annotate the individual electronic clinical documents. In certain aspects, annotating the training documents includes, but is not limited to, digitally highlighting or marking various specific text within each clinical document that, if present, corresponds to or supports the presence of each of the various clinical indicators. In certain aspects, the annotation further includes, but is not limited to, thereafter tagging each instance of the highlighted text with an appropriate label that corresponds to the clinical indicator, or category of clinical indicator, for which the text was highlighted.

In addition, in certain aspects, synthetic text and/or data, and its corresponding clinical indicator tags may also be generated, via the recommendations service 12, on the at least one computing device 10 by the clinical experts to use as additional training data input to the AI service 14. In this manner, the AI service 14 can further be trained to identify numerous possible variations on the specific strings of text, phrasing, written natural language, and/or data that the AI service 14 may encounter in future clinical documents, and be able to apply the appropriate clinical indicators, or categories of clinical indicators, thereto. For example, the at least one computing device 10 via expert clinician input, can generate, via the recommendation service 12, as inputs to the AI service 14, multiple sentences that each have varying phrasing, but which all map to the same clinical indicator “IV Antibiotics.” This additional synthetic data is input into the AI service 14 as further training documents in order to train the AI service 14 to capture the many text and written natural language permutations that can be used to indicate or describe in a patient's medical records that the patient will need continued “IV Antibiotics.”

The annotated clinical documents generated by the at least one computing device 10, containing the highlighted/marked text and their applied clinical indicator tags, are, via the recommendation service 12, transmitted to or inputted into the AI service 14 to train the neural network 34, for example, of the AI service 14 to recognize the specific highlighted text and variations/permutations thereof, and automatically associate such text with specific clinical indicator tag(s) applied thereto by the clinical experts. In this manner, the AI service 14 is learning that specific text and phrases in the clinical documents of a patient's medical records indicate the presence of different clinical indicators. And these clinical indicators will be used in the recommendation logic of the recommendation service 12 to either support, or weigh against, a recommendation of admission of a patient to a SNF for post-acute care.

### Clinical Indicators

In certain aspects, the clinical indicators identified by the at least one computing device 10, via the recommendation service 12, that the AI service 14 is trained to identify fall into, but not limited to, three different categories: nursing indicators, therapy indicators, and counter indicators. In other aspects, there may be fewer or additional categories of clinical indicators that the AI service 14 has been trained to identify, without departing from the scope of the present disclosure.

The nursing indicators are a category of clinical indicators that describe a patient's need for daily skilled nursing, and if present in a patient's clinical documents of their medical records, support a recommendation for SNF admission. The specific clinical indicators that fall within the nursing indicators category, and which the AI service 14 is trained, via the recommendation service 12, to identify, include, but is not limited to, complex wound care indicators, intravenous (“IV”) antibiotics indicators, feeding tube indicators, ventilator indicators, tracheostomy indicators, and other appropriate nursing indicators. The complex wound care indicator would be applied to any text, phrasing, or data present in a patient's clinical documents that would signify that the patient requires daily care for complex wounds, which care would not be possible or safe to administer at home, or which daily care would not be as effective if administered elsewhere without skilled nursing. The IV antibiotics indicators would be applied to any text, phrasing, or data in a patient's clinical documents that would signify that the patient requires the administration of at least daily IV antibiotics for an infection, which IV antibiotics would be difficult to administer at home or without skilled nursing care. In certain aspects, with respect to the IV antibiotics indicators, the AI service 14 is trained, via the recommendation service 12, to look for diagnoses of osteomyelitis and endocarditis in the patient's medical records as part of the criteria for the AI service 14 to indicate or trigger the application of the IV antibiotics indicator to the patient clinical document. As an example, the feeding tube indicator would be applied to any text, phrasing, or data that would signify a patient has a feeding tube that requires skilled nursing care at a SNF, and also requires receiving enteral feeding via the feeding tube that comprises at least the minimum percentage of daily nutrition requirements per day.

The therapy indicators are a category of clinical indicators that describe a patient's need for physical and/or occupational therapy at a SNF, and if present in a patient's medical records, tends to support a recommendation for SNF admission. The specific clinical indicator that falls within the therapy indicators category includes at least a decline in level of function (“LOF”) indicator. In order for a patient to require therapy at a SNF, the clinical criteria articulates that a patient must have experienced a meaningful or significant drop/decline in their LOF for a given physical skill, as compared to their baseline LOF. Accordingly, for a given physical skill or function, a decline in LOF exceeding a threshold amount tends to support a recommendation for SNF admission. To ascertain if there has been a significant decline in a patient's LOF, a determination must be made of (1) a patient's current level of function (“CLOF”) as of the time the patient is to be discharged from the hospital after acute care and admitted to a SNF, (2) the patient's prior level of function (“PLOF”) prior to the need for, or receipt of, the acute care (i.e, the baseline LOF), and (3) a difference between the PLOF and the CLOF.

For each PLOF and CLOF needed to make an assessment of the amount of decline in LOF for a patient, clinical experts highlight the text in clinical documents related to descriptions of a patient's LOF and tag the patient's clinical documents with a description that details what the LOF was (i.e. PLOF) and what the LOF is now (i.e. CLOF). Generating the description involves specifying values for each of three variables for each of the two types of LOF: (1) the type of LOF being described (i.e. CLOF or PLOF); (2) the skill (or physical function) being assessed/evaluated; (3) and a description of the patient's level of ability, or level of assistance needed, for that function or skill.

For example, in certain aspects, a patient's clinical data in their medical records may include a doctor or therapist note related to post treatment observations of a patient's gait that states, “The patient's current level of function demonstrates a moderate assist level of ambulatory function, with noticeable limitations in gait dynamics.” The clinical expert will highlight or annotate the text in the clinical document, via the at least one computing device 10, and tag or label the document for each of the three variables associated with the level of function indicator, as mentioned above, that indicate the value of each of the three variables.

For example:
1. LOF Type: CLOF
2. Skill Assessed: Gait
3. LOF Description: Moderate Assistance

These labels may be recorded by generating a table, database, or dictionary of “key” and “value” pairs for each CLOF and PLOF, in which the keys are function or skill categories (e.g. sit-to-stand, gait, etc.), and the “value” is the LOF description (e.g. independent, moderate assistance, total dependence, etc.). In order to find if there is a decline in a patient's LOF for a given skill, both of the CLOF and PLOF must be determined for that assessed skill and tagged in the patient's clinical data. Numeric values are also assigned to each of the specific CLOF and PLOF descriptions for a given skill, based on the description of the patient's level of ability or assistance needed for that function or skill. The numeric value of the PLOF description is subtracted from the numeric value of the CLOF description for that skill to arrive at a decline in LOF numeric value or score. If the resulting calculated decline in LOF value meets or exceeds a threshold value, this constitutes a significant decline in LOF sufficient to apply the decline in LOF indicator to the patient's file, and to support admission to a SNF for continued therapy.

In certain aspects, the numeric values assigned to each of the LOF descriptions for a given assessed skill may be as follows:

TABLE 1
Exemplary Table illustrating possible LOF Descriptions
and associated assigned numeric values

| | LOF Description | Numeric Value |
|---|---|---|
| 1. | Complete Independence | 0 |
| 2. | Modified Independence | 0 |
| 3. | Supervision or Setup (Stand-By Assist) | 0 |
| 4. | CGA (Contact Guard Assist) | 0 |
| 5. | Minimal Assistance | 2 |
| 6. | Moderate Assistance | 3 |
| 7. | Maximal Assistance | 4 |
| 8. | Total Dependence | 5 |

In such aspects, for example, a threshold value for measuring the level of decline in an assessed skill may be set at ≥2 such that if the numeric value of CLOF−PLOF≥2, this is considered a significant decline in LOF, which tends to support admission to a SNF for continued post-acute care or therapy. Continuing with the example above, and with the LOF descriptions and assigned numeric values in table 1 above, assume that the CLOF and PLOF for a patient's walking gait are able to be determined from a patient's clinical documents as follows:

| 1. LOF Type: | CLOF | PLOF |
|---|---|---|
| 2. Skill Assessed: | Gait | Gait |
| 3. LOF Description: | Moderate Assistance | Complete Independence |
| 4. Numeric Value: | 3 | 0 |

In this example, when the decline in LOF calculation meets or exceeds the predetermined threshold value of ≥2, the patient's file will be tagged with a decline in LOF indicator. In the above example, CLOF=3 and PLOF=0, such that CLOF−PLOF=3−0=3. And since 3≥2 (the threshold value), this patient's clinical file would be tagged with the decline in LOF indicator. The AI service 14 is trained to make such calculations. However, in other aspects, the LOF descriptions may be different, or there may be fewer or additional LOF descriptions, or the numeric values assigned to each of the LOF descriptions may be different than those disclosed in the table and examples above without departing from the scope of the present disclosure.

Because both PLOF and CLOF tags are required to arrive at a calculated value corresponding to the amount of decline in a patient's LOF, if a patient's clinical documents do not include text, phrasing, and/or data from which an assessment or tagging of a patients PLOF or CLOF can be made, then it is not possible to make an assessment as to, or calculate an amount of, a decline in LOF. In this case, admission of the patient to a SNF for therapy cannot be recommended.

The counter indicators are a category of clinical indicators that counter, or weigh against, recommending admission to a SNF. The counter indicators signify the presence of a situation, factor, condition, or consideration in a patient's clinical file that counteracts the presence of therapy indicators in the patient's clinical documents, and accordingly their presence either minimizes, or completely negates, the benefits that a patient would otherwise gain if admitted to a SNF for therapy. The specific clinical indicators that fall within the counter indicators category, and which the AI service 14 is trained, via the recommendation service 12, to identify in a patient's medical records, include a long-term care indicator, an impaired cognition indicator, and a home caregiver indicator.

The long-term care indicator signifies that the patient was admitted to a long-term care facility prior to being admitted to the hospital for acute care. The long-term care indicator counteracts the presence of therapy indicators because the patient's needs may already be met in the long-term care setting with 24-hours custodial support, thus minimizing any benefits that might be realized by SNF level of care. The impaired cognition indicator signifies that the patient has some level of impaired mental cognition. This impaired cognition may inhibit the patient's participation in, and therefore any perceived benefit received from, physical or occupational therapy that would be administered in a SNF. In annotating the clinical documents on the at least one computing device 10 to train the AI service 14, via the recommendation service 12, to recognize text indicative of the impaired cognition indicator, the clinical experts specifically look for any details regarding the patients' inability to follow instructions as part of the trigger for applying the impaired cognition indicator label.

The home caregiver indicator signifies that the patient has adequate care support from a caregiver in the patient's home, and therefore the patient's post-acute care service needs may be adequately provided for at a lower level of care in the home, as opposed to those provided in a SNF. In annotating the clinical documents on the at least one computing device 10 to train the AI service 14, via the recommendation service 12, to recognize text indicative of the home caregiver indicator, the clinical experts are looking for clear indication that the patient was previously receiving help from someone at home, and that the level of support given in the home is still adequate to continue to do so with the patient's post-acute care needs.

In certain aspects, the AI service 14 may include a single learning processor (e.g., the neural network 34) that is trained, and thereafter used, to identify the various clinical indicators, or categories of clinical indicators, in the clinical documents of a patient's medical records as discussed above. In other aspects, the AI service 14 may include two AI models, three AI model, or more separate AI models, each with their own separate learning processors, that are trained and thereafter used to identify various of the clinical indicators in each category, without departing for the scope of the present disclosure.

In certain aspects, the AI service 14 is trained by the recommendation service 12 to identify and indicate the presence or absence of nursing indicators and/or counter indicators (or the specific clinical indicators in each such category), while a second AI model is trained to identify the presence or absence of therapy indicators (or the specific clinical indicators within this category). In other aspects, the AI service 14 may include a first AI model trained to identify the presence or absence of nursing indicators (or the specific clinical indicators therein), a second AI model trained to identify the presence or absence of therapy indicators (or the specific clinical indicators therein), and a third AI model trained to identify the presence or absence counter indicators (or the specific clinical indicators therein).

The AI models used in the analysis system of the present disclosure can include any one or more of a natural language processing model, a large language model (a “LLM”), a deep neural network model, logistic regression model, decision tree model, language representation model, or any other appropriate type of AI model, or combinations thereof.

In certain aspects, the training inputs generated by the recommendation service 12 that are used to train the AI service 14 are clinical documents from a number of patient medical records retrieved from the electronic medical records service 16, for example, and the natural language text and/or data contained therein. In such aspects, once the AI service 14 has been trained, the specific clinical documents of a patient to be discharged from a hospital are used as inputs for the AI service 14 to identify for that patient as to which clinical indicators or categories of clinical indicators are present in that patient's clinical documents. The outputs from the AI service 14, after a patient's clinical documents have been inputted and analyzed by the AI model, are received by the recommendation service 12 and can include the generation of true/false data, tags, labels, or tables, that document the presence each of the relevant clinical indicators, or categories of clinical indicators, in the patient's clinical document(s). The true/false data specifies whether each such clinical indicator, for which the AI service 14 is scanning the clinical document, is present (i.e. a “true” indication) in a given clinical document or is absent (i.e. a “false” indication) from a given clinical document(s). Other forms of output from the AI service 14 that track the presence of each clinical indicator in a patient's clinical document, may be generated, tracked, or stored, without departing from the scope of the present disclosure.

After the AI service 14 is trained by the recommendation service 12 to recognize various text in the clinical documents of patient medical records and apply the proper clinical indictors corresponding thereto, the AI service 14 can then be used to quickly and objectively analyze the clinical documents in the medical records of other patients who are to be discharged from the hospital, and need a recommendation as to whether to be discharged to a SNF for further post-acute care.

To do so the AI service 14 is in communication, either directly or via the recommendation service 12, with a hospitals electronic medical records system (e.g., the electronic medical records service 16), and able to access clinical documents of a patient to be discharged from the hospital or other acute care medical facility. The clinical documents of the patient to be discharged are first transmitted or loaded into the AI service 14, as the input for the analysis to be completed. The clinical documents are then scanned by the AI service 14 to search for the specific text, phrasing, data, and variations thereof that it was trained to identify corresponding to the various clinical indicators or categories of clinical indicators discussed above. In certain aspects, the AI service 14 then generates true/false data corresponding to the presence of each such clinical indicator or category of clinical indicator found in the clinical documents, which can be received by the recommendation service 12. A designation of “true” for a clinical indicator indicates the clinical indicator is present in the clinical documents being analyzed, while a designation of “false” indicates that the clinical indicator is not present in the clinical documents. In other aspects, the AI service 14 may generate this data in another form or format, such as a table that indicates each clinical indicator and whether or not it is present in the clinical file, without departing from the scope of the present disclosure. In still other aspects, the AI service 14 may simply apply tags to the clinical file that indicate only the specific clinical indicators that the AI model deems are present in the clinical document, and for any remaining clinical indicators that don't have tags applied to the clinical document such clinical indicators are deemed by default not to be present in the clinical document. Once the AI service 14 scans the clinical document and generates the output data, in whatever form, that identifies which clinical indicators are present in the clinical document and which are absent, the output data is sent or transmitted to the recommendation service 12 to make an ultimate recommendation as to admission of the patient to a SNF. In certain aspects, the AI service 14 receives scans of the clinical document from the first computing device 10a.

### Decision/Recommendation Logic

The recommendation service 12 further includes, for example, a recommendation logic programmed therein to make a recommendation regarding admission of a given patient to a SNF, based on the clinical indicators output data identified by the AI service 14 in a patient's clinical documents.

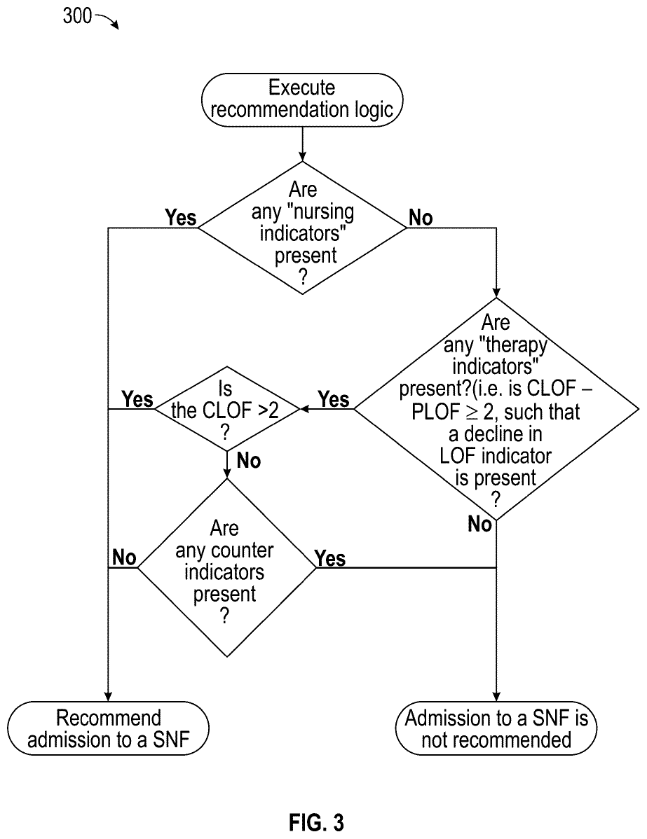

Referring to FIG. 3, the recommendation logic of the recommendation service 12 is executed after the AI service 14 generates the respective outputs as disclosed above, and applies a decision tree structure logic to those outputs to arrive at a final recommendation to be output to the patient's payor, insurer, or other such third party. In certain aspects, the logic works as follows.

The recommendation service 12 searches the output data received from the AI service 14 to determine whether any nursing indicators, therapy indicators, or counter indicators are present in the patient's clinical data. If any nursing indicators are present, regardless of the presence of any therapy indicators or counter indicators, the recommendation logic and thus the recommendation service 12 outputs a final recommendation that the patient should be admitted to SNF for continued post-acute care.

If no such nursing indicators are present in the patient clinical document, the recommendation logic of the recommendation service 12 next checks to see if there are any therapy indicators present in the patient's clinical documents. By way of reminder, in certain aspects, the presence of a therapy indicator in the clinical documents is indicative that a decline in LOF indicator is present. In certain aspects, the recommendation service 12 may have already made this determination as discussed above by calculating whether the numeric value of the CLOF minus the numeric value of the PLOF exceeds a threshold amount. In certain aspects, that threshold amount is ≥2, such that for any CLOF−PLOF≥2, a decline LOF indicator is present in the patient's clinical document. The threshold value for determining whether a decline in LOF is present may vary in different embodiments without departing from the scope of the present disclosure. In other aspects, the recommendation service 12 may simply tag/document each of the CLOF and PLOF values without calculating the difference between the CLOF and PLOF values, and those CLOF and PLOF values are evaluated by the recommendations service 12 which then calculates whether the numeric value of the CLOF−PLOF exceeds the threshold amount. In either scenario, if the calculated decline in LOF as CLOF−PLOF exceeds the threshold amount, then a decline in LOF indicator is present, which also means that a therapy indicator is present as well in the patient's clinical document.

Thus, returning to the recommendation logic of the recommendation service 12, if no nursing indicators are present, and no therapy indicators are present, the recommendation logic and thus the recommendation service 12 outputs a final recommendation that the patient should be not be admitted to SNF, as there is most likely no clinical need for SNF. However, if no nursing indicators are present, but a therapy indicator is present, then the recommendation service 12 next checks to see if the CLOF is greater than the threshold value, such as for example, whether CLOF>2.

If the CLOF is greater than the threshold value (e.g. CLOF>2) then the recommendation logic and thus the recommendation service 12 outputs a final recommendation that the patient should be admitted to SNF for continued post-acute care. If however, the CLOF is not greater than the threshold value, such as for example the CLOF≤2, then the recommendation logic next checks to see if there are any counter indicators present in the patient's clinical documents.

If there are no counter indicators present in the patient patient's clinical documents, then the recommendation logic and thus the recommendation service 12 outputs a final recommendation that the patient should be admitted to SNF for continued post-acute care. However, if there are counter indicators present, the recommendation logic and thus the recommendation service 12 outputs a final recommendation that the patient should not be admitted to SNF, as the counter indicators minimize or negate any benefit the patient might otherwise receive from being admitted to SNF for continued care.

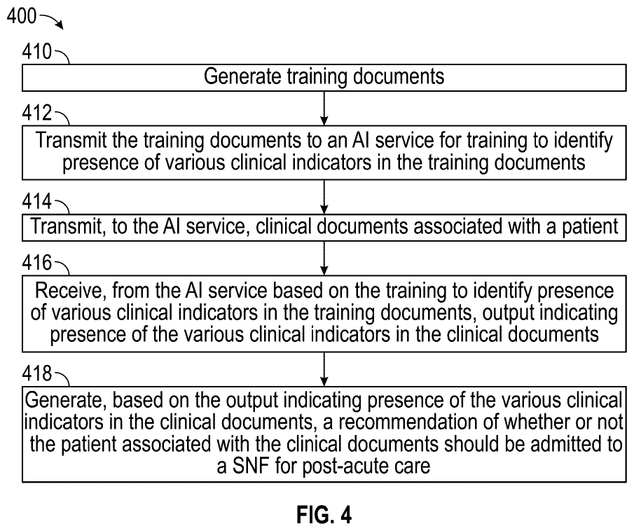

FIG. 4 illustrates an example process 400 using the recommendation service 12 and the AI service 14 of FIG. 2. It should be understood that the process steps of FIG. 4 may be performed by other systems, as described herein.

The process 400 begins by proceeding to step 410 when the processor 28 of the recommendation service 12 generates training documents. As depicted at step 412, the processor 28 of the recommendation service 12 transmits the training documents to an AI service 14 for training to identify presence of various clinical indicators in the training documents. As depicted at step 414, the processor 28 of the recommendation service 12 transmits, to the AI service 14, clinical documents associated with a patient. The processor 28 of the recommendation service 12 receives, from the AI service 14 based on the training to identify presence of various clinical indicators in the training documents, output indicating presence of the various clinical indicators in the clinical documents, as depicted at step 416. As depicted at step 418, the processor 28 of the recommendation service 12 generates, based on the output indicating presence of the various clinical indicators in the clinical documents, a recommendation of whether or not the patient associated with the clinical documents should be admitted to a SNF for post-acute care.

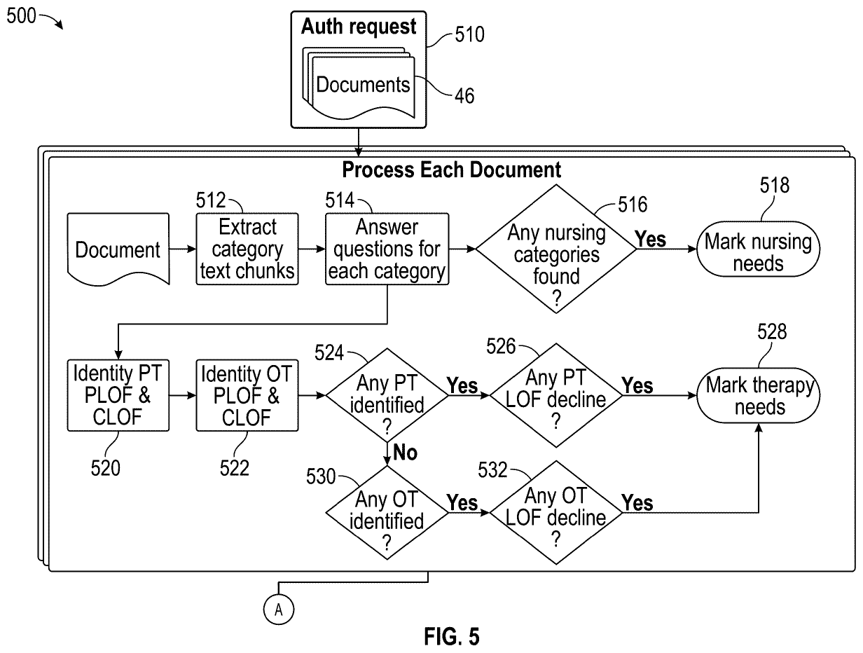

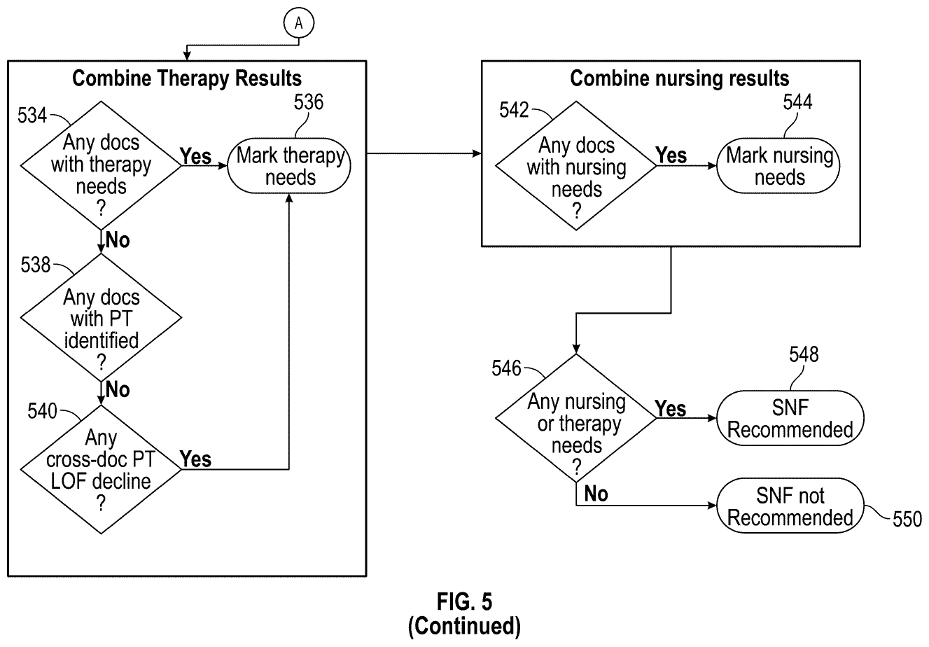

FIG. 5 illustrates another example process for generating a post-acute care recommendation using the first computing device 10a, the AI service 14, and the electronic medical records service 16 of FIG. 2. While FIG. 5 Is described with reference to FIG. 2, it should be understood that the process steps of FIG. 5 may be performed by other systems.

The process 500 begins by proceeding to step 510 when the recommendation service 12 receives an authorization request for a patient to be admitted to a skilled nursing facility. As illustrated at step 512, the AI service 14 receives clinical documents 46 associated with the patient from the recommendation service 12 and/or the electronic medical records service 16. For each document of the clinical documents 46 associated with the patient, the AI service 14, via the recommendation service 12, identifies or extracts text associated with any of the clinical categories from the document, such as, but not limited to, the nursing indicators categories and the therapy indicators categories. For example, the AI service 14, via the recommendation service 12, identifies or extracts text associated with any of the clinical categories from each document of the clinical documents 46 such as the nursing indicators categories including, but not limited to, complex wound care indicators, intravenous (“IV”) antibiotics indicators, feeding tube indicators, ventilator indicators, tracheostomy indicators, and other appropriate nursing indicators, and such as the therapy indicators categories including, but not limited to, PLOF and CLOF for both physical therapy and for occupational therapy based on skills such as, but not limited to, transfers, gait, ambulation, and other appropriate skills.

Subsequent to identifying or extracting text associated with any of the clinical categories from each document of the clinical documents 46, the AI service 14, via the recommendation service 12, proceeds to append source data (e.g., where the document was sourced such as from, but not limited to, the patient, family of the patient, a physical therapist of the patient, an occupational therapist of the patient, a speech therapist of the patient, a physician of the patient, a nurse of the patient, and other appropriate sources) and date of the document to each of the identified or extract text. The date of the document is the observation date for the identified or extracted text. In certain aspects, when the date of the document cannot be determined, the date of the document is approximated based on print date of the document or a prepared date of the document. In certain aspects, the date of the document is used to restrict the identified or extracted text associated with physical therapy CLOF and occupational therapy CLOF to only those within 3 days of the authorization request date. In certain aspects, the source data is used to restrict which identified or extracted text are used for physical therapy and occupational therapy evaluations.

As illustrated at step 514, the AI service 14, via the recommendation service 12, proceeds to query each of the identified or extracted text, associated with any of the clinical categories, with detection questions particular to each of the nursing indicators categories and the therapy indicators categories. For example, with the ventilator indicators category of the nursing indicators categories, the AI service 14, via the recommendation service 12, queries the identified or extracted text with detection questions such as, but not limited to, “Does the patient currently have a ventilator?,” “Is the date the patient's ventilator was placed known?,” “Was the patient's ventilator placed within 30 days of the Authorization Request Date?,” and other appropriate detection questions. For example, with the IV antibiotics indicators category of the nursing indicators categories, the AI service 14, via the recommendation service 12, queries the identified or extracted text with detection questions such as, but not limited to, “Is the patient currently receiving IV antibiotics?,” “Are the patient's IV antibiotic needs at least daily?,” “Is the duration or end date of the patient's IV antibiotics known?,” “Are the patient's IV antibiotics to be administered for more than 3 days after the Authorization Request Date?,” and other appropriate detection questions. For example, with the wound care indicators of the nursing indicators categories, the AI service 14, via the recommendation service 12, queries the identified or extracted text with detection questions such as, but not limited to, “Does the patient currently have wound care needs?,” “Are the patient's wound care needs at least daily?,” “Is the duration or end date of the patient's wound care known?,” “Will the patient require wound care after discharge?,” “Are the patient's wound care needs complex such that they require packing or debriding?,” and other appropriate detection questions. For example, with tracheostomy indicators of the nursing indicators categories, the AI service 14, via the recommendation service 12, queries the identified or extracted text with detection questions such as, but not limited to, “Does the patient currently have a tracheostomy?,” “Is the date the patient's tracheostomy was placed known?,” “Was the patient's tracheostomy placed within 30 days of the Auth Request Date?,” “Does the patient's tracheostomy require close daily monitoring?,” and other appropriate detection questions. For example, with feeding tube indicators of the nursing indicators categories, the AI service 14, via the recommendation service 12, queries the identified or extracted text with detection questions such as, but not limited to, “Does the patient currently have a feeding tube?,” “Is the date the patient's feeding tube was placed known?,” “Was the patient's feeding tube placed within 30 days of the Auth Request Date?,” “Is the patient receiving at least 25% of their calories from the feeding tube?,” and other appropriate detection questions.

As illustrated at step 516, the AI service 14, via the recommendation service 12, determines whether any nursing indicators categories are found. For example, when all of the detection questions for a particular nursing indicators category are answered affirmatively that particular nursing indicators category (e.g., the identified or extracted text) is marked as “found,” as depicted at step 518. It should be understood that the AI service 14, via the recommendation service 12, is configured to mark the identified or extracted text with other labels based on determined criteria. For example, a label of “insufficient” is marked when the AI service 14, via the recommendation service 12, determines that enough information was found, but it does not meet the clinical criteria for daily skilled nursing care; a label of “partial” when the AI service 14, via the recommendation service 12, determines that some, but not all, of the information necessary to make a decision was found (e.g., if any of the questions for a category could not be answered, the result will be “partial,” even if some of the questions were answered, even if some of the questions were answered negatively); and a label of “missing” when the AI service 14, via the recommendation service 12, determines that none of the information necessary to make a decision was found.

In addition to querying each of the identified or extracted text, associated with any of the clinical categories, with detection questions, the AI service 14, via the recommendation service 12, also identifies, from the identified or extracted text and based on the skills (e.g., transfers, gait, ambulation), PLOF and CLOF for physical therapy indicators and occupational therapy indicators of the therapy indicators categories, as depicted at step 520 and step 522, respectively. At step 524, the AI service 14, via the recommendation service 12, determines whether any physical therapy indicators were identified. If physical therapy indicators were identified, then the AI service 14, via the recommendation service 12, determines whether there is a decline in physical therapy LOF per the approach described above, as depicted at step 526. When the AI service 14, via the recommendation service 12, determines that there is a decline in physical therapy LOF, the AI service 14, via the recommendation service 12, marks the identified or extracted text as identifying therapy needs, as depicted at step 528.

Moving back to step 524, if the AI service 14, via the recommendation service 12, determines that there were no physical therapy indicators identified, then the AI service 14, via the recommendation service 12, determines whether any occupational therapy indicators were identified, as depicted at step 530. If occupational therapy indicators were identified, then the AI service 14, via the recommendation service 12, determines whether there is a decline in occupational therapy LOF per the approach described above, as depicted at step 532. When the AI service 14, via the recommendation service 12, determines that there is a decline in occupational therapy LOF, the AI service 14, via the recommendation service 12, marks the identified or extracted text as identifying therapy needs, as depicted at step 528. That is to say, the identified or extracted text is marked as identifying a decline in function if a decline in function was identified from a physical therapy LOF, or a decline in function was identified from an occupational therapy LOF and there was not enough physical therapy indicators to make a decision.

In certain other aspects, the numeric values assigned to each of the LOF descriptions for a given assessed skill may be as follows:

| | LOF Description | Numeric Value |
|---|---|---|
| 1. | Complete Independence | −1 |
| 2. | Modified Independence | −1 |
| 3. | Supervision or Setup (Stand-By Assist) | −1 |
| 4. | CGA (Contact Guard Assist) | −1 |
| 5. | Minimal Assistance | 2 |
| 6. | Moderate Assistance | 3 |
| 7. | Maximal Assistance | 4 |
| 8. | Total Dependence | 5 |

In certain aspects, in addition to the approach described above for determining whether there is a decline in physical therapy LOF and occupational therapy LOF, both physical therapy LOF and occupational therapy LOF decline is defined as a difference between the highest PLOF skill rating score and the lowest CLOF skill rating score of at least 2. In cases where the lowest PLOF skill rating was dependent (e.g., equal to 5), however, the lowest PLOF skill rating is used to calculate the difference instead of the highest PLOF skill rating. In certain aspects, when calculating the CLOF skill ratings, the identified or extracted text are only used if they are from a date within 3 days of the authorization request date, however, if less than 30%, for example, of the identified or extract text include a date, then all the identified or extracted text is considered without filtering by date. In certain aspects, only identified or extracted text with a source of “physical therapist” are used for physical therapy CLOF. In certain aspects, only identified or extracted text with a source of “occupational therapist” are used for occupational therapy CLOF. In certain aspects, identified or extracted text associated with occupational therapy are only used when there is not enough physical therapy information to make a decision. If physical therapy indicators are found, but there is no decline identified by the physical therapy, then occupational therapy is not considered. If physical therapy LOF indicates a decline, then occupational therapy is not considered.

In certain aspects, if no single document has enough physical therapy information to make a decision, and no single document identified a decline in occupational therapy LOF, then the highest physical therapy PLOF and lowest physical therapy CLOF from across all the clinical documents 46 are used to identify a decline in function. Only the most recent PLOF and CLOF values are used.

With each document reviewed, the AI service 14, via the recommendation service 12, determines whether any of the identified or extracted text are marked as identifying therapy needs based on physical therapy, as depicted at step 534. Based on determining that any one of the identified or extracted text is marked as identifying therapy needs based on physical therapy, the AI service 14, via the recommendation service 12, marks the identified or extracted text as having therapy needs, as depicted at step 536. On the other hand, when the AI service 14, via the recommendation service 12, determines that none of the identified or extracted text is marked as identifying therapy needs, the AI service 14, via the recommendation service 12, queries the identified or extracted text to determine whether any of the identified or extracted text is identified with physical therapy indicators, as depicted at step 538. When the AI service 14, via the recommendation service 12, determines that none of the identified or extracted text is identified with physical therapy indicators, the AI service 14, via the recommendation service 12, queries the identified or extracted text to determine whether any identified or extracted text identifies physical therapy LOF, as depicted at step 540. When the AI service 14, via the recommendation service 12, determines that one of the identified or extracted text identifies physical therapy LOF, the AI service 14, via the recommendation service 12, marks the identified or extracted text as having therapy needs, as depicted at step 536.

As depicted at step 542, the AI service 14, via the recommendation service 12, then determines whether any of the identified or extracted text is marked as “found” for including a nursing indicators category. Based on determining that any of the identified or extracted text is marked as “found,” the AI service 14, via the recommendation service 12, marks the identified or extracted text as having nursing needs, as depicted at step 544.

As depicted at step 546, the AI service 14, via the recommendation service 12, then determines whether any of the identified or extracted text is marked with having nursing needs or therapy needs. Based on determining that one of the identified or extracted text is marked with having nursing needs or therapy needs, the recommendation service 12, generates a recommendation for SNF admission, as depicted at step 548. On the other hand, when the recommendation service 12, determines that none of the identified or extracted text is marked with having nursing needs or therapy needs, the AI service 14, via the recommendation service 12, does not recommend SNF admission, as depicted at step 550. In certain aspects, the recommendation service 12, generates results including, but not limited to, an explanation of the recommendation, a listing of labeling of the clinical documents 46 and/or identified or extracted text by “found,” “partial,” “missing,” and “insufficient,” for example.

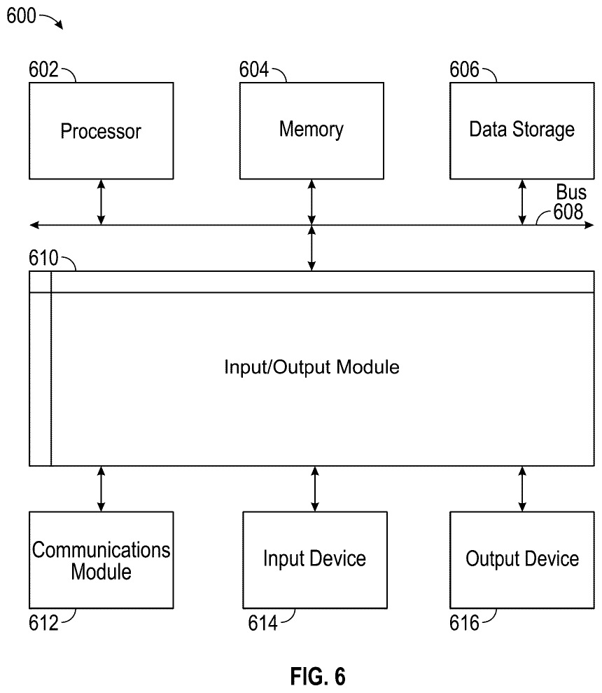

FIG. 6 is a block diagram illustrating an example computer system 600 with which the at least one computing device 10, such as the first computing device 10a, the recommendation service 12, the AI service 14, and the electronic medical records service 16 of FIG. 2 can be implemented. In certain aspects, the computer system 600 may be implemented using hardware or a combination of software and hardware, either in a dedicated server, or integrated into another entity, or distributed across multiple entities.

Computer system 600 (e.g., the at least one computing device 10, such as the first computing device 10a, the recommendation service 12, the AI service 14, and the electronic medical records service 16) includes a bus 608 or other communication mechanism for communicating information, and a processor 602 (e.g., the processor 28, 32, 38, 42) coupled with bus 608 for processing information. According to one aspect, the computer system 600 can be a cloud computing server of an IaaS that is able to support PaaS and SaaS services.

Computer system 600 can include, in addition to hardware, code that creates an execution environment for the computer program in question, e.g., code that constitutes processor firmware, a protocol stack, a database management system, an operating system, or a combination of one or more of them stored in an included memory 604 (e.g., the memory 30, 36, 40, 44), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 608 for storing information and instructions to be executed by processor 602. The processor 602 and the memory 604 can be supplemented by, or incorporated in, special purpose logic circuitry.

The instructions may be stored in the memory 604 and implemented in one or more computer program products, e.g., one or more modules of computer program instructions encoded on a computer readable medium for execution by, or to control the operation of, the computer system 600.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 600 further includes a data storage device 606 such as a magnetic disk or optical disk, coupled to bus 608 for storing information and instructions. Computer system 600 may be coupled via input/output module 610 to various devices. The input/output module 610 can be any input/output module. Example input/output modules 610 include data ports such as USB ports. In addition, input/output module 610 may be provided in communication with processor 602, so as to enable near area communication of computer system 600 with other devices. The input/output module 610 may provide, for example, for wired communication in some implementations, or for wireless communication in other implementations, and multiple interfaces may also be used. The input/output module 610 is configured to connect to a communications module 612. Example communications modules 612 (e.g., the communications module 20, 22, 24, 26) include networking interface cards, such as Ethernet cards and modems.

In certain aspects, the input/output module 610 is configured to connect to a plurality of devices, such as an input device 614 and/or an output device 616. Example input devices 614 include a keyboard and a pointing device, e.g., a mouse or a trackball, by which a user can provide input to the computer system 600. Other kinds of input devices 614 can be used to provide for interaction with a user as well, such as a tactile input device, visual input device, audio input device, or brain-computer interface device.

According to one aspect of the present disclosure the at least one computing device 10, such as the first computing device 10a, the recommendation service 12, the AI service 14, and the electronic medical records service 16 can be implemented using a computer system 600 in response to processor 602 executing one or more sequences of one or more instructions contained in memory 604. Such instructions may be read into memory 604 from another machine-readable medium, such as data storage device 606. Execution of the sequences of instructions contained in main memory 604 causes processor 602 to perform the process steps described herein. One or more processors in a multi-processing arrangement may also be employed to execute the sequences of instructions contained in memory 604. Processor 602 may process the executable instructions and/or data structures by remotely accessing the computer program product, for example by downloading the executable instructions and/or data structures from a remote server through communications module 612 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

The term “machine-readable storage medium” or “computer-readable medium” as used herein refers to any medium or media that participates in providing instructions or data to processor 602 for execution. The term “storage medium” as used herein refers to any non-transitory media that store data and/or instructions that cause a machine to operate in a specific fashion. Such a medium may take many forms, including, but not limited to, non-volatile media, volatile media, and transmission media.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 608. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

In one aspect, a method may be an operation, an instruction, or a function and vice versa. In one aspect, a clause or a claim may be amended to include some or all of the words (e.g., instructions, operations, functions, or components) recited in either one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

What is claimed is:

1. A computer-implemented method for generating a post-acute care recommendation comprising:
generating training documents;
transmitting the training documents to an AI service for training to identify presence of various clinical indicators in the training documents;
transmitting, to the AI service, clinical documents associated with a patient;
receiving, from the AI service based on the training to identify presence of various clinical indicators in the training documents, output indicating presence of the various clinical indicators in the clinical documents; and
generating, based on the output indicating presence of the various clinical indicators in the clinical documents, a recommendation of whether or not the patient associated with the clinical documents should be admitted to a SNF for post-acute care.

2. The computer-implemented method of claim 1, wherein transmitting, to the AI service, clinical documents associated with a patient is based on receiving an authorization request.

3. The computer-implemented method of claim 1, further comprising extracting text from each document of the clinical documents associated with the patient based on clinical categories.

4. The computer-implemented method of claim 3, wherein the clinical categories comprise nursing indictor categories and therapy indicators categories.

5. The computer-implemented method of claim 3, further comprising appending source data and date of document to the text extracted from each document.

6. The computer-implemented method of claim 4, further comprising marking the text extracted from each document with “found” based on identifying a nursing indicators category being answered affirmatively.

7. The computer-implemented method of claim 1, further comprising generating results comprising an explanation of the recommendation.

8. A system comprising:
one or more memories comprising instructions; and
one or more processors configured to execute the instructions, which, when executed, cause the one or more processors to:
generate training documents;
transmit the training documents to an AI service for training to identify presence of various clinical indicators in the training documents;
transmit, to the AI service, clinical documents associated with a patient;
receive, from the AI service based on the training to identify presence of various clinical indicators in the training documents, output indicating presence of the various clinical indicators in the clinical documents; and
generate, based on the output indicating presence of the various clinical indicators in the clinical documents, a recommendation of whether or not the patient associated with the clinical documents should be admitted to a SNF for post-acute care.

9. The system of claim 8, wherein the one or more processors are configured to execute instructions, which when executed, cause the one or more processors to transmit, to the AI service, clinical documents associated with a patient based on receiving an authorization request.

10. The system of claim 8, wherein the one or more processors are further configured to execute instructions, which when executed, cause the one or more processors to extract text from each document of the clinical documents associated with the patient based on clinical categories.

11. The system of claim 10, wherein the clinical categories comprise nursing indictor categories and therapy indicators categories.

12. The system of claim 10, wherein the one or more processors are further configured to execute instructions, which when executed, cause the one or more processors to append source data and date of document to the text extracted from each document.

13. The system of claim 11, wherein the one or more processors are further configured to execute instructions, which when executed, cause the one or more processors to mark the text extracted from each document with “found” based on identifying a nursing indicators category being answered affirmatively.

14. The system of claim 8, wherein the one or more processors are further configured to execute instructions, which when executed, cause the one or more processors to generate results comprising an explanation of the recommendation.

15. A non-transitory machine-readable storage medium comprising machine-readable instructions for causing one or more processors to execute a method, the method comprising:
generating training documents;
transmitting the training documents to an AI service for training to identify presence of various clinical indicators in the training documents;
transmitting, to the AI service, clinical documents associated with a patient;
receiving, from the AI service based on the training to identify presence of various clinical indicators in the training documents, output indicating presence of the various clinical indicators in the clinical documents; and
generating, based on the output indicating presence of the various clinical indicators in the clinical documents, a recommendation of whether or not the patient associated with the clinical documents should be admitted to a SNF for post-acute care.

16. The non-transitory machine-readable storage medium of claim 15, further comprising extracting text from each document of the clinical documents associated with the patient based on clinical categories.

17. The non-transitory machine-readable storage medium of claim 15, wherein the clinical categories comprises nursing indictor categories and therapy indicators categories.

18. The non-transitory machine-readable storage medium of claim 16, further comprising appending source data and date of document to the text extracted from each document.

19. The non-transitory machine-readable storage medium of claim 17, further comprising marking the text extracted from each document with “found” based on identifying a nursing indicators category being answered affirmatively.

20. The non-transitory machine-readable storage medium of claim 15, further comprising generating results comprising an explanation of the recommendation.
